(19) United States
(12) Patent Application Publication
(10) Pub. No.: US 2022/0389088 A1
(43) Pub. Date: Dec. 8, 2022

(54) ANTI-ANGIOPOIETIN-LIKE 4 (ANGPTL4) ANTIBODIES AND METHODS OF USE

(71) Applicant: UNIVERSITY OF FLORIDA RESEARCH FOUNDATION, INCORPORATED, Gainesville, FL (US)

(72) Inventors: Weizhou Zhang, Gainesville, FL (US); Ryan Harrison Kolb, Gainesville, FL (US)

(21) Appl. No.: 17/771,339

(22) PCT Filed: Oct. 29, 2020
(86) PCT No.: PCT/US2020/057964

§ 371 Date: Apr. 22, 2022

Related U.S. Application Data
(60) Provisional application No. 62/927,289, filed on Oct. 29, 2019.

(51) Int. Cl. C07K 16/22; A61P 35/00; A61K 45/06; A61K 39/395

(52) U.S. Cl. CPC C07K 16/22; A61P 35/00; A61K 45/06; A61K 39/3955; C07K 2317/21; C07K 2317/24; C07K 2317/55; C07K 2317/622; C07K 2317/92; A61K 2039/505

(57) ABSTRACT

Described herein are anti-angiopoictin-like 4 (ANGPTL4) antibodies and methods of treating disorders associated with elevated expression of ANGPTL4. In one aspect, described herein is an antibody or antigen binding fragment thereof that binds angiopoietin-like 4 (ANGPTL4) comprising amino acid sequences set forth in (a) SEQ ID NOs: 3-8 (Ab-A CDRs), (b) SEQ ID NOs: 13-18 (Ab-B CDRs), (c) SEQ ID NOs: 23-28 (Ab-C CDRs), (d) SEQ ID NOs: 33-38 (Ab-D CDRs), or (e) SEQ ID NOs: 43-48 (Ab-E CDRs).

### CROSS-REFERENCE TO RELATED APPLICATION

The present application claims the benefit of priority to U.S. Provisional Application No. 62/927,289, filed Oct. 29, 2019, the disclosure of which is incorporated herein by reference in its entirety.

### STATEMENT OF GOVERNMENT SUPPORT

This invention was made with government support under grant No. CA200673 awarded by the National Institutes of Health. The government has certain rights in the invention.

### INCORPORATION BY REFERENCE

This application contains, as a separate part of the disclosure, a sequence listing in computer-readable form (filename: 54753_SeqListing.txt Size: 28,936 bytes; Created: Oct. 28, 2020), which is incorporated by reference in its entirety.

### BACKGROUND

Obesity is associated with an increased risk of estrogen receptor-positive breast cancer in postmenopausal women and a worse clinical outcome regardless of menopausal status (Kolb et al., Curr. Opin. Pharmacol., 29:77-89, 2016). While outcomes for obese patients are worse, treatment options are the same despite often being less efficacious due to dose limiting toxicities and obesity-related complications, thus underlying the need to develop specific therapies for better treating obese patients. An association between obesity and increased tumor angiogenesis has been reported in several other studies (Gu et al., Cancer Biol. Ther. 11:910-917, 2011; Fukumursa et al., Microcirculation, 23:191-206, 2016; Arendt et al., Cancer Res., 73:6080-6093, 2013) underscoring the potential importance of increased angiogenesis to obesity-driven breast cancer progression.

### SUMMARY

In one aspect, described herein is an antibody or antigen binding fragment thereof that binds angiopoietin-like 4 (ANGPTL4) comprising amino acid sequences set forth in (a) SEQ ID NOs: 3-8 (Ab-A CDRs), (b) SEQ ID NOs: 13-18 (Ab-B CDRs), (c) SEQ ID NOs: 23-28 (Ab-C CDRs), (d) SEQ ID NOs: 33-38 (Ab-D CDRs), or (e) SEQ ID NOs: 43-48 (Ab-E CDRs). In some embodiments, the antibody or antigen binding fragment comprises a light chain variable region at least 90% identical to an amino acid sequence set forth in (a) SEQ ID NO: 10 (Ab-A LC variable), (b) SEQ ID NO: 20 (Ab-B LC variable), (c) SEQ ID NO: 30 (Ab-C LC variable), (d) SEQ ID NO: 40 (Ab-D LC variable), or (e) SEQ ID NO: 50 (Ab-E LC variable). In some embodiments, antibody or antigen binding fragment comprises a light chain variable region comprising an amino acid sequence set forth in (a) SEQ ID NO: 10 (Ab-A LC variable), (b) SEQ ID NO: 20 (Ab-B LC variable), (c) SEQ ID NO: 30 (Ab-C LC variable), (d) SEQ ID NO: 40 (Ab-D LC variable), or (e) SEQ ID NO: 50 (Ab-E LC variable). In some embodiments, antibody or antigen binding fragment comprises a heavy chain variable region at least 90% identical to an amino acid sequence set forth in (a) SEQ ID NO: 9 (Ab-A HC variable), (b) SEQ ID NO: 19 (Ab-B HC variable), (c) SEQ ID NO: 29 (Ab-C HC variable), (d) SEQ ID NO: 39 (Ab-D HC variable), or (e) SEQ ID NO: 49 (Ab-E HC variable). In some embodiments, antibody or antigen binding fragment comprises a heavy chain variable region comprising an amino acid sequence set forth in (a) SEQ ID NO: 9 (Ab-A HC variable), (b) SEQ ID NO: 19 (Ab-B HC variable), (c) SEQ ID NO: 29 (Ab-C HC variable), (d) SEQ ID NO: 39 (Ab-D HC variable), or (e) SEQ ID NO: 49 (Ab-E HC variable).

In some embodiments, the antibody or antigen binding fragment thereof is a monoclonal antibody. In some embodiments, the antibody or antigen binding fragment thereof is a human or humanized antibody. In some embodiments, the antibody or antigen binding fragment thereof is an IgG. In some embodiments, the antigen binding fragment thereof is a Fab fragment or an scFv.

In another aspect, a method of inhibiting ANGPLT4-induced angiogenesis in subject in need thereof, comprising administering to the subject the antibody or antigen binding fragment described herein is also contemplated. In some embodiments, the subject is suffering from cancer. In some embodiments, the subject is suffering from breast cancer or renal cell carcinoma. In some embodiments, the breast cancer is basal-like breast cancer. In some embodiments, the subject is obese. In some embodiments, the subject is suffering from diabetic retinopathy. In some embodiments, the subject is suffering from macular age-related degeneration (AMD). In some embodiments, the AMD is wet AMD. In some embodiments, the subject was hyporesponsive to prior anti-VEGF-A therapy. Exemplary anti-VEGF-A therapies include, but are not limited to, sorafenib, sunitinib, bevacizumab and ranibizumab. In some embodiments, the subject was resistant to treatment with sorafenib, sunitinib, bevacizumab and/or ranibizumab.

In another aspect, a method of treating cancer in a subject in need thereof comprising administering to the subject the antibody or antigen binding fragment thereof described herein is also contemplated. In some embodiments, the cancer is breast cancer or renal cell carcinoma. In some embodiments, the breast cancer is basal-like breast cancer. In some embodiments, the subject is obese. In some embodiments, the method further comprises administering chemotherapy to the subject.

In another aspect, a method of treating a disorder associated with elevated angiopoietin-like 4 (ANGPTL4) in a subject in need thereof comprising administering to the subject an antibody or antigen binding fragment described herein and an anti-VEGF-A therapy is also contemplated. In some embodiments, the disorder associated with elevated ANGPTL4 is cancer, diabetic retinopathy or age-related macular degeneration (AMD). In some embodiments, the cancer is breast cancer or renal cell carcinoma. In some embodiments, the breast cancer is basal-like breast cancer. In some embodiments, the age-related macular degeneration is wet AMD. In some embodiments, the subject is obese. In some embodiments, the VEGF-A therapy is sorafenib, sunitinib, bevacizumab or ranibizumab. In some embodiments, the subject was resistant to treatment with sorafenib, sunitinib, bevacizumab and/or ranibizumab.

### BRIEF DESCRIPTION OF THE FIGURES

FIGS. 1A and 1B show the results of an in vitro assay for anti-cANGPTL4 antibody inhibition of cANGPTL4 induced angiogenesis. Endothelial cells were cultured with recombinant human cANGPTL4 in the presence and absence of the indicated rat-anti-human cANGPTL4 antibody and tube formation (FIG. 1B) was assessed.

FIG. 2 shows the results of the in vivo matrigel plug assay to assess the ability of Antibody Ab-D to inhibit cANGPTL4-induced angiogenesis.

FIG. 3 is a graph showing the expression of ANGPTL4 in human cancer.

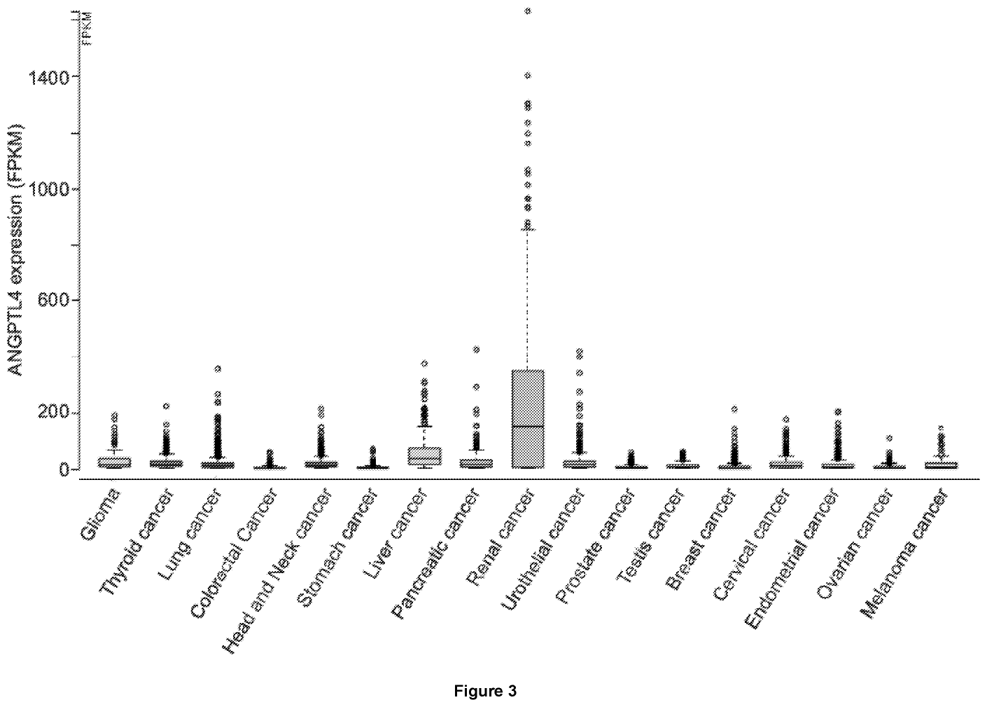

FIG. 4 is a graph showing that tumor volume was decreased in a mouse model of renal cell carcinoma treated with the anti-ANGPTL4 antibody compared to the control.

FIG. 5 are graphs showing increased tumor immune cell infiltration, including T cells, in a mouse model of renal cell carcinoma treated with anti-ANGPTL4 antibody compared to the control.

FIG. 6 are graphs showing that ANGPTL4 expression is upregulated in tumor resistant to treatment with sorafenib or sunitinib.

### DETAILED DESCRIPTION

Described herein are monoclonal antibodies against the c-terminus of ANGPTL4 (cANGPTL4) that are capable of inhibiting the induction of angiogenesis by cANGPTL4. Angiopoietin-like 4 (ANGPTL4) is a secreted protein that is cleaved into two active peptides (Lei et al., J. Biol. Chem., 286:15747-15756, 2011). The N-terminus domain is a potent inhibitor of lipoprotein lipase (LPL) activity and modulates lipid composition and energy homeostasis (Lei et al., supra). The C-terminus (cANGPTL4) domain is involved in wound healing, vessel permeability, and angiogenesis (Zhu et al., Biosci. Rep., 32:211-219, 2012).

Antibodies

The disclosure also provides anti-angiopoietin-like 4 (ANGPTL4) antibodies. The term “antibody” refers to an intact immunoglobulin molecule (including polyclonal, monoclonal, chimeric, humanized, and/or human versions having full length heavy and/or light chains). The antibody may be any type of antibody, i.e., immunoglobulin, known in the art. In exemplary embodiments, the antibody is an antibody of class or isotype IgA, IgD, IgE, IgG, or IgM. In exemplary embodiments, the antibody described herein comprises one or more alpha, delta, epsilon, gamma, and/or mu heavy chains. In exemplary embodiments, the antibody described herein comprises one or more kappa or light chains. In exemplary aspects, the antibody is an IgG antibody and optionally is one of the four human subclasses: IgG1, IgG2, IgG3 and IgG4. Also, the antibody in some embodiments is a monoclonal antibody. In other embodiments, the antibody is a polyclonal antibody. In some aspects, the antibody is a chimeric or a humanized antibody. The term “humanized” when used in relation to antibodies refers to antibodies having at least CDR regions from a non-human source and which are engineered to have a structure and immunological function more similar to true human antibodies than the original source antibodies. For example, humanizing can involve grafting CDRs from a non-human antibody, such as a mouse antibody, into a human antibody framework. Humanizing also can involve select amino acid substitutions to make a non-human sequence look more like a human sequence.

In some aspects, the antibody is a Humaneered™ antibody. Humaneering technology converts non-human antibodies into engineered human antibodies. Humaneered™ antibodies have high affinity, and are highly similar to human germline antibody sequences. See, e.g., Tomasevic et al., Growth Factors 32: 223-235 (2014).

“Specifically binds” as used herein means that the antibody (or antigen binding fragment) preferentially binds an antigen (e.g., ANGPTL4) over other proteins. In some embodiments, “specifically binds” means the antibody has a higher affinity for the antigen than for other proteins. Antibodies that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. It will be appreciated that ranges having the values above as end points is contemplated in the context of the disclosure. For example, the antibody or antigen binding fragment thereof may bind ANGPTL4 of SEQ ID NO: 1 with an affinity of about 1×10⁻⁷ M to about 9×10⁻¹² M or an affinity of 1×10⁻⁹ to about 9×10⁻¹².

In some or any embodiments, the antibody (or antigen binding fragment) binds to ANGPTL4 of SEQ ID NO: 1, or a naturally occurring variant thereof, with an affinity (Kd) of less than or equal to 1×10⁻⁷ M, less than or equal to 1×10⁻⁸M, less than or equal to 1×10⁻⁹ M, less than or equal to 1×10⁻¹⁰ M, less than or equal to 1×10⁻¹¹ M, or less than or equal to 1×10⁻¹² M, or ranging from 1×10⁻⁹ to 1×10⁻¹⁰, or ranging from 1×10⁻¹² to about 1×10⁻¹³. Affinity is determined using a variety of techniques, examples of which include an affinity ELISA assay and a surface plasmon resonance (BIAcore) assay.

In some or any embodiments, the antibody (or antigen binding fragment thereof) binds to a ANGPTL4 peptide comprising amino acids 166-406 of SEQ ID NO: 1 (Genbank Accession No. Q9BY76), with any of the affinities described above.

“CDR” refers to the complementarity determining region within antibody variable sequences. There are three CDRs in each of the variable regions of the heavy chain and the light chain, which are designated CDR1, CDR2 and CDR3, for each of the variable regions. The term “set of six CDRs” as used herein refers to a group of three CDRs that occur in the light chain variable region and heavy chain variable region, which are capable of binding the antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs are obtained by, e.g., constructing polynucleotides that encode the CDR of interest and expression in a suitable host cell. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 3, CDR-H2 has the sequence given in SEQ ID NO: 4, CDR-H3 has the sequence given in SEQ ID NO: 5, CDR-L1 has the sequence given in SEQ ID NO: 6, CDR-L2 has the sequence given in SEQ ID NO: 7 and CDR-L3 has the sequence given in SEQ ID NO: 8. In various aspects, the antibody (or antigen binding fragment thereof) comprises a CDR-H1 having the sequence given in SEQ ID NO: 3 with 3, 2, or 1 amino acid substitutions therein, CDR-H2 having the sequence given in SEQ ID NO: 4 with 3, 2, or 1 amino acid substitutions therein, CDR-H3 having the sequence given in SEQ ID NO: 5 with 3, 2, or 1 amino acid substitutions therein, CDR-L1 having the sequence given in SEQ ID NO: 6 with 3, 2, or 1 amino acid substitutions therein, CDR-L2 having the sequence given in SEQ ID NO: 7 with 3, 2, or 1 amino acid substitutions therein and CDR-L3 having the sequence given in SEQ ID NO: 8 with 3, 2, or 1 amino acid substitutions therein. The anti-ANGPTL4 antibody, in various aspects, comprises two of the CDRs, three of the CDRs, four of the CDRs, five of the CDRs or all six of the CDRs. In a preferred embodiment, the anti-ANGPTL4 antibody comprises a set of six CDRs as follows: CDR-H1 of SEQ ID NO: 3, CDR-H2 of SEQ ID NO: 4, CDR-H3 of SEQ ID NO: 5, CDR-L1 of SEQ ID NO: 6, CDR-L2 of SEQ ID NO: 7 and CDR-L3 of SEQ ID NO: 8.

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 13, CDR-H2 has the sequence given in SEQ ID NO: 14, CDR-H3 has the sequence given in SEQ ID NO: 15, CDR-L1 has the sequence given in SEQ ID NO: 16, CDR-L2 has the sequence given in SEQ ID NO: 17 and CDR-L3 has the sequence given in SEQ ID NO: 18. In various aspects, the antibody (or antigen binding fragment thereof) comprises a CDR-H1 having the sequence given in SEQ ID NO: 13 with 3, 2, or 1 amino acid substitutions therein, CDR-H2 having the sequence given in SEQ ID NO: 14 with 3, 2, or 1 amino acid substitutions therein, CDR-H3 having the sequence given in SEQ ID NO: 15 with 3, 2, or 1 amino acid substitutions therein, CDR-L1 having the sequence given in SEQ ID NO: 16 with 3, 2, or 1 amino acid substitutions therein, CDR-L2 having the sequence given in SEQ ID NO: 17 with 3, 2, or 1 amino acid substitutions therein and CDR-L3 having the sequence given in SEQ ID NO: 18 with 3, 2, or 1 amino acid substitutions therein. The anti-ANGPTL4 antibody, in various aspects, comprises two of the CDRs, three of the CDRs, four of the CDRs, five of the CDRs or all six of the CDRs. In a preferred embodiment, the anti-ANGPTL4 antibody comprises a set of six CDRs as follows: CDR-H1 of SEQ ID NO: 13, CDR-H2 of SEQ ID NO: 14, CDR-H3 of SEQ ID NO: 15, CDR-L1 of SEQ ID NO: 16, CDR-L2 of SEQ ID NO: 17 and CDR-L3 of SEQ ID NO: 18.

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 23, CDR-H2 has the sequence given in SEQ ID NO: 24, CDR-H3 has the sequence given in SEQ ID NO: 25, CDR-L1 has the sequence given in SEQ ID NO: 26, CDR-L2 has the sequence given in SEQ ID NO: 27 and CDR-L3 has the sequence given in SEQ ID NO: 28. In various aspects, the antibody (or antigen binding fragment thereof) comprises a CDR-H1 having the sequence given in SEQ ID NO: 23 with 3, 2, or 1 amino acid substitutions therein, CDR-H2 having the sequence given in SEQ ID NO: 24 with 3, 2, or 1 amino acid substitutions therein, CDR-H3 having the sequence given in SEQ ID NO: 25 with 3, 2, or 1 amino acid substitutions therein, CDR-L1 having the sequence given in SEQ ID NO: 26 with 3, 2, or 1 amino acid substitutions therein, CDR-L2 having the sequence given in SEQ ID NO: 27 with 3, 2, or 1 amino acid substitutions therein and CDR-L3 having the sequence given in SEQ ID NO: 28 with 3, 2, or 1 amino acid substitutions therein. The anti-ANGPTL4 antibody, in various aspects, comprises two of the CDRs, three of the CDRs, four of the CDRs, five of the CDRs or all six of the CDRs. In a preferred embodiment, the anti-ANGPTL4 antibody comprises a set of six CDRs as follows: CDR-H1 of SEQ ID NO: 23, CDR-H2 of SEQ ID NO: 24, CDR-H3 of SEQ ID NO: 25, CDR-L1 of SEQ ID NO: 26, CDR-L2 of SEQ ID NO: 27 and CDR-L3 of SEQ ID NO: 28.

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 33, CDR-H2 has the sequence given in SEQ ID NO: 34, CDR-H3 has the sequence given in SEQ ID NO: 35, CDR-L1 has the sequence given in SEQ ID NO: 36, CDR-L2 has the sequence given in SEQ ID NO: 37 and CDR-L3 has the sequence given in SEQ ID NO: 38. In various aspects, the antibody (or antigen binding fragment thereof) comprises a CDR-H1 having the sequence given in SEQ ID NO: 33 with 3, 2, or 1 amino acid substitutions therein, CDR-H2 having the sequence given in SEQ ID NO: 34 with 3, 2, or 1 amino acid substitutions therein, CDR-H3 having the sequence given in SEQ ID NO: 35 with 3, 2, or 1 amino acid substitutions therein, CDR-L1 having the sequence given in SEQ ID NO: 36 with 3, 2, or 1 amino acid substitutions therein, CDR-L2 having the sequence given in SEQ ID NO: 37 with 3, 2, or 1 amino acid substitutions therein and CDR-L3 having the sequence given in SEQ ID NO: 38 with 3, 2, or 1 amino acid substitutions therein. The anti-ANGPTL4 antibody, in various aspects, comprises two of the CDRs, three of the CDRs, four of the CDRs, five of the CDRs or all six of the CDRs. In a preferred embodiment, the anti-ANGPTL4 antibody comprises a set of six CDRs as follows: CDR-H1 of SEQ ID NO: 33, CDR-H2 of SEQ ID NO: 34, CDR-H3 of SEQ ID NO: 35, CDR-L1 of SEQ ID NO: 36, CDR-L2 of SEQ ID NO: 37 and CDR-L3 of SEQ ID NO: 38.

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 43, CDR-H2 has the sequence given in SEQ ID NO: 44, CDR-H3 has the sequence given in SEQ ID NO: 45, CDR-L1 has the sequence given in SEQ ID NO: 46, CDR-L2 has the sequence given in SEQ ID NO: 47 and CDR-L3 has the sequence given in SEQ ID NO: 48. In various aspects, the antibody (or antigen binding fragment thereof) comprises a CDR-H1 having the sequence given in SEQ ID NO: 43 with 3, 2, or 1 amino acid substitutions therein, CDR-H2 having the sequence given in SEQ ID NO: 44 with 3, 2, or 1 amino acid substitutions therein, CDR-H3 having the sequence given in SEQ ID NO: 45 with 3, 2, or 1 amino acid substitutions therein, CDR-L1 having the sequence given in SEQ ID NO: 46 with 3, 2, or 1 amino acid substitutions therein, CDR-L2 having the sequence given in SEQ ID NO: 47 with 3, 2, or 1 amino acid substitutions therein and CDR-L3 having the sequence given in SEQ ID NO: 48 with 3, 2, or 1 amino acid substitutions therein. The anti-ANGPTL4 antibody, in various aspects, comprises two of the CDRs, three of the CDRs, four of the CDRs, five of the CDRs or all six of the CDRs. In a preferred embodiment, the anti-ANGPTL4 antibody comprises a set of six CDRs as follows: CDR-H1 of SEQ ID NO: 43, CDR-H2 of SEQ ID NO: 44, CDR-H3 of SEQ ID NO: 45, CDR-L1 of SEQ ID NO: 46, CDR-L2 of SEQ ID NO: 47 and CDR-L3 of SEQ ID NO: 48.

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NOs: 10, 20, 30, 40 or 50 and/or a heavy chain variable region comprising an amino acid sequence having at least 75% identity (e.g., at least 75%, 80%, 85%, 90%, 95% or 100% identity) to the amino acid sequence set forth in SEQ ID NOs: 9, 19, 29, 39, or 49. In various aspects, the difference in the sequence compared to SEQ ID NO: 10, 20, 30, 40, or 50 (or SEQ ID NOs: 9, 19, 29, 39 or 49) lies outside the CDR region in the corresponding sequences.

In some or any embodiments, the antibody (or antigen binding fragment) comprises a light chain variable region comprising an amino acid sequence set forth in SEQ ID NO: 10 and a heavy chain variable region comprising an amino acid sequence set forth in SEQ ID NO: 9. In some or any embodiments, the antibody (or antigen binding fragment) comprises a light chain variable region encoded by the nucleotide sequence set forth in SEQ ID NO: 12. In some or any embodiments, the antibody (or antigen binding fragment) comprises a heavy chain variable region encoded by the nucleotide sequence set forth in SEQ ID NO: 11.

In some or any embodiments, the antibody (or antigen binding fragment) comprises a light chain variable region comprising an amino acid sequence set forth in SEQ ID NO: 20 and a heavy chain variable region comprising an amino acid sequence set forth in SEQ ID NO: 19. In some or any embodiments, the antibody (or antigen binding fragment) comprises a light chain variable region encoded by the nucleotide sequence set forth in SEQ ID NO: 22. In some or any embodiments, the antibody (or antigen binding fragment) comprises a heavy chain variable region encoded by the nucleotide sequence set forth in SEQ ID NO: 21.

In some or any embodiments, the antibody (or antigen binding fragment) comprises a light chain variable region comprising an amino acid sequence set forth in SEQ ID NO: 30 and a heavy chain variable region comprising an amino acid sequence set forth in SEQ ID NO: 29. In some or any embodiments, the antibody (or antigen binding fragment) comprises a light chain variable region encoded by the nucleotide sequence set forth in SEQ ID NO: 32. In some or any embodiments, the antibody (or antigen binding fragment) comprises a heavy chain variable region encoded by the nucleotide sequence set forth in SEQ ID NO: 31.

In some or any embodiments, the antibody (or antigen binding fragment) comprises a light chain variable region comprising an amino acid sequence set forth in SEQ ID NO: 40 and a heavy chain variable region comprising an amino acid sequence set forth in SEQ ID NO: 39. In some or any embodiments, the antibody (or antigen binding fragment) comprises a light chain variable region encoded by the nucleotide sequence set forth in SEQ ID NO: 42. In some or any embodiments, the antibody (or antigen binding fragment) comprises a heavy chain variable region encoded by the nucleotide sequence set forth in SEQ ID NO: 41.

In some or any embodiments, the antibody (or antigen binding fragment) comprises a light chain variable region comprising an amino acid sequence set forth in SEQ ID NO: 50 and a heavy chain variable region comprising an amino acid sequence set forth in SEQ ID NO: 49. In some or any embodiments, the antibody (or antigen binding fragment) comprises a light chain variable region encoded by the nucleotide sequence set forth in SEQ ID NO: 52. In some or any embodiments, the antibody (or antigen binding fragment) comprises a heavy chain variable region encoded by the nucleotide sequence set forth in SEQ ID NO: 51.

Antigen binding fragments of the anti-ANGPTL4 antibodies described herein are also contemplated. The antigen binding fragment can be any part of an antibody that has at least one antigen binding site, and the antigen binding fragment may be part of a larger structure (an “antibody product”) that retains the ability of the antigen binding fragment to recognize ANGPTL4. For ease of reference, these antibody products that include antigen binding fragments are included in the disclosure herein of “antigen binding fragment.” Examples of antigen binding fragments, include, but are not limited to, Fab, F(ab′)₂, a monospecific or bispecific Fab₂, a trispecific Fab₃, scFv, dsFv, scFv-Fc, bispecific diabodies, trispecific triabodies, minibodies, a fragment of IgNAR (e.g., V-NAR), a fragment of hcIgG (e.g., VhH), bis-scFvs, fragments expressed by a Fab expression library, and the like. In exemplary aspects, the antigen binding fragment is a domain antibody, VhH domain, V-NAR domain, VH domain, VL domain, or the like. Antibody fragments of the disclosure, however, are not limited to these exemplary types of antibody fragments. In exemplary aspects, antigen binding fragment is a Fab fragment. In exemplary aspects, the antigen binding fragment comprises two Fab fragments. In exemplary aspects, the antigen binding fragment comprises two Fab fragments connected via a linker. In exemplary aspects, the antigen binding fragment comprises or is a minibody comprising two Fab fragments. In exemplary aspects, the antigen binding fragment comprises, or is, a minibody comprising two Fab fragments joined via a linker. Minibodies are known in the art. See, e.g., Hu et al., Cancer Res 56: 3055-3061 (1996). In exemplary aspects, the antigen binding fragment comprises or is a minibody comprising two Fab fragments joined via a linker, optionally, comprising an alkaline phosphatase domain.

A domain antibody comprises a functional binding unit of an antibody, and can correspond to the variable regions of either the heavy (VH) or light (VL) chains of antibodies. A domain antibody can have a molecular weight of approximately 13 kDa, or approximately one-tenth of a full antibody. Domain antibodies may be derived from full antibodies such as those described herein.

In some embodiments, the scFv is attached to a human Fc domain. In some embodiments, the Fc domain does not activate Fc effector functions.

### Methods of Antibody or Antigen Binding Fragment Production

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5th Ed., Garland Publishing, New York, N.Y. (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1). Antibodies may also be produced by inducing in vivo production in the lymphocyte population or by screening recombinant immunoglobulin libraries or panels of highly specific binding reagents as disclosed in Orlandi et al (Proc Natl Acad Sci 86: 3833-3837; 1989), and Winter G and Milstein C (Nature 349: 293-299, 1991). If the full sequence of the antibody or antigen-binding fragment is known, then methods of producing recombinant proteins may be employed. See, e.g., “Protein production and purification” Nat Methods 5(2): 135-146 (2008). In some embodiments, the antibodies (or antigen binding fragments) are isolated from cell culture or a biological sample if generated in vivo.

Phage display also can be used to generate the antibody of the present disclosures. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; 5,702,892. The techniques described in U.S. Pat. Nos. 5,780,279; 5,821,047; 5,824,520; 5,855,885; 5,858,657; 5,871,907; 5,969,108; 6,057,098; and 6,225,447.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol, 235, 959-973 (1994). A preferred chimeric or humanized antibody has a human constant region, while the variable region, or at least a CDR, of the antibody is derived from a non-human species. Methods for humanizing non-human antibodies are well known in the art. (See, e.g., U.S. Pat. Nos. 5,585,089, and 5,693,762.)

Techniques developed for the production of “chimeric antibodies,” e.g., the splicing of mouse antibody genes to human antibody genes to obtain a molecule with appropriate antigen specificity and biological activity, can be used (Morrison et al., Proc Natl Acad Sci 81: 6851-6855 (1984); Neuberger et al., Nature 312: 604-608 (1984); Takeda et al., Nature 314: 452-454 (1985)). Alternatively, techniques described for the production of single chain antibodies (U.S. Pat. No. 4,946,778) can be adapted to produce ANGPTL4-specific single chain antibodies.

Likewise, using techniques known in the art to isolate CDRs, compositions comprising CDRs are generated. Compositions comprising one, two, and/or three CDRs of a heavy chain variable region or a light chain variable region of a monoclonal antibody can be generated. The CDRs of exemplary antibodies are provided herein as SEQ ID NOs: 4-9 and 12-17. Techniques for cloning and expressing nucleotide and polypeptide sequences are well-established in the art (see, e.g., Sambrook et al., Molecular Cloning: A Laboratory Manual, 2nd Edition, Cold Spring Harbor, N.Y. (1989)). The amplified CDR sequences are ligated into an appropriate expression vector. The vector comprising one, two, three, four, five and/or six cloned CDRs optionally contains additional polypeptide encoding regions linked to the CDR.

Chemically constructed bispecific antibodies may be prepared by chemically cross-linking heterologous Fab or F(ab′)2 fragments by means of chemicals such as heterobifunctional reagent succinimidyl-3-(2-pyridyldithiol)-propionate (SPDP, Pierce Chemicals, Rockford, Ill.). The Fab and F(ab′)2 fragments can be obtained from intact antibody by digesting it with papain or pepsin, respectively (Karpovsky et al., J. Exp. Med. 160:1686-701 (1984); Titus et al., J. Immunol., 138:4018-22 (1987)).

Methods of testing antibodies for the ability to bind to an epitope of ANGPTL4, regardless of how the antibodies are produced, are known in the art and include, e.g., radioimmunoassay (RIA), ELISA, Western blot, immunoprecipitation, surface plasmon resonance (e.g., BIAcore), and competitive inhibition assays (see, e.g., Janeway et al., infra, and U.S. Patent Application Publication No. 2002/0197266).

Antibody fragments that contain the antigen binding, or idiotype, of the antibody molecule may be generated by techniques known in the art. For example, a F(ab′)2 fragment may be produced by pepsin digestion of the antibody molecule; Fab′ fragments may be generated by reducing the disulfide bridges of the F(ab′)2 fragment; and two Fab′ fragments which may be generated by treating the antibody molecule with papain and a reducing agent. The disclosure is not limited to enzymatic methods of generating antigen binding fragments; the antigen binding fragment may be a recombinant antigen binding fragment produced by expressing a polynucleotide encoding the fragment in a suitable host cell.

A single-chain variable region fragments (scFv), which consists of a truncated Fab fragment comprising the variable (V) domain of an antibody heavy chain linked to a V domain of an antibody light chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

### Detection Methods

It is sometimes desirable to detect the presence or measure the amount of ANGPTL4 in a sample. In this regard, the disclosure provides a method of using the antibody or fragment thereof described herein to measure the amount of ANGPTL4 in a sample. To determine a measurement of ANGPTL4, a biological sample from a mammalian subject is contacted with an anti-ANGPTL4 antibody (or antigen binding fragment thereof) described herein for a time sufficient to allow immunocomplexes to form. Immunocomplexes formed between the antibody and ANGPTL4 in the sample are then detected. The amount of ANGPTL4 in the biological sample is optionally quantitated by measuring the amount of the immunocomplex formed between the antibody and the ANGPTL4. For example, the antibody can be quantitatively measured if it has a detectable label, or a secondary antibody can be used to quantify the immunocomplex.

In some embodiments, the biological sample comprises a tissue sample, a cell sample, or a biological fluid sample, such as blood, saliva, serum, or plasma.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats can readily be adapted to employ the antibodies (or fragments thereof) of the present disclosure. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test sample used in the above-described method will vary based on the assay format, nature of the detection method and the tissues, cells or fluids used as the sample to be assayed.

The assay described herein may be useful in, e.g., evaluating the efficacy of a particular therapeutic treatment regime in animal studies, in clinical trials, or in monitoring the treatment of an individual patient.

In some embodiments, anti-ANGPTL4 antibody (or antigen binding fragment thereof) is attached to a solid support, and binding is detected by detecting a complex between the ANGPTL4 and the antibody (or antigen binding fragment thereof) on the solid support. The antibody (or fragment thereof) optionally comprises a detectable label and binding is detected by detecting the label in the ANGPTL4-antibody complex.

Detection of the presence or absence of a ANGPTL4-antibody complex be achieved using any method known in the art. For example, the transcript resulting from a reporter gene transcription assay of a ANGPTL4 peptide interacting with a target molecule (e.g., antibody) typically encodes a directly or indirectly detectable product (e.g., β-galactosidase activity and luciferase activity). For cell free binding assays, one of the components usually includes, or is coupled to, a detectable label. A wide variety of labels can be used, such as those that provide direct detection (such as radioactivity, luminescence, optical or electron density) or indirect detection (such as epitope tag such as the FLAG epitope, enzyme tag such as horseradish peroxidase). The label can be bound to the antibody, or incorporated into the structure of the antibody.

A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers or indirectly detected with antibody conjugates, or streptavidin-biotin conjugates. Methods for detecting the labels are well known in the art.

### Therapeutic Methods

In another aspect, described herein is a method of treating a disorder associated with elevated angiopoietin-like 4 (ANGPTL4), the method comprising administering to the subject an antibody or antigen binding fragment thereof. Exemplary disorders associated with elevated ANGPTL4 include, but are not limited to, cancer, diabetic retinopathy age-related macular degeneration.

A. Cancer

In some embodiments, the disorder associated with elevated ANGPTL4 is cancer. Exemplary cancers include, but are not limited to, breast cancer, renal cell carcinoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, richter's syndrome; waldenstrom macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteo sarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and recurrent mycosis fungoides and Sezary syndrome.

In some embodiments, an anti-ANGPTL4 antibody described herein is administered to a subject suffering from breast cancer or renal cell carcinoma. In some embodiments, an anti-ANGPTL4 antibody described herein is administered to a subject suffering from basal-like breast cancer.

It is contemplated that the methods herein reduce tumor burden, and/or reduce metastasis in the subject, and/or reduce or prevent the recurrence of tumors once the cancer has gone into remission. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

In some embodiments, the subject to be treated is hyporesponsive to treatment with an anti-VEGF-A therapy (e.g., the subject has failed to respond or responded sub-optimally to previous anti-VEGF-A therapy). In some embodiments, the subject is obese.

B. Diabetic Retinopathy

Diabetic retinopathy (DR) is a major cause of vision problems affecting 93 million people worldwide⁹. DR is associated with inflammation and increased neovascularization which can lead to vitreous hemorrhage, retinal detachment or neovascular glaucoma¹⁰. Anti-VEGF therapies have been reported to be effective in treating DR, however may patients fail to respond or only have a partial response¹¹. Several studies have shown that ANGPTL4 levels were elevated in the vitreous and serum of patients with DR, and that inhibition of ANGPTL4 could reduce the angiogenic potential of aqueous fluid from patients with low levels of VEGFA or who had received anti-VEGFA therapy^{9, 12}. The use of anti-ANGPTL4 antibodies described herein for the treatment of diabetic retinopathy (either alone or in combination with anti-VEGFA therapy) is specifically contemplated. In some embodiments, the VEGF-A therapy is reanibizumab.

C. Age-Related Macular Degeneration

Age-related macular degeneration (AMD) is one of the most common causes of vision impairment in elderly people. Neovascularization is associated with “wet” AMD and ranibizumab, an anti-VEGFA antibody, has been approved for the treatment of AMD. Ranibizumab has been shown to improve vision in wet AMD patients but requires continual treatment and frequent intravitreal injections. Studies have found that ANGPTL4 levels were elevated in wet AMD patients and were correlated with lesion area¹³. Thus, the treatment of wet AMD with anti-ANGPTL4 antibodies is specifically contemplated.

D. Combination Therapy

In any of the therapeutic methods described herein, administering an anti-VEGF-A therapy in combination with an anti-ANGPTL4 antibody described herein is specifically contemplated. Exemplary anti-VEGF-A therapies include, but are not limited to, sorafenib, sunitinib, bevacizumab and ranibizumab.

Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In various embodiments, an anti-ANGPTL4 antibody or antigen binding fragment thereof described herein is administered in combination with an additional therapeutic useful for treating cancer. The additional therapeutic may be other therapeutic agents, such as cytokines, growth factors, other inhibitors and antibodies to target antigens useful for treating cancer or immunological disorders, for example ipilimumab (YERVOY®, Bristol-Myers Squibb Company), an antibody to CTLA-4; bevacizumab (AVASTIN®, Genentech), an antibody to VEGF-A; erlotinib (TARCEVA®, Genentech and OSI Pharmaceuticals), a tyrosine kinase inhibitor which acts on EGFR, dasatinib (SPRYCEL®, Bristol-Myers Squibb Company), an oral Bcr-Abl tyrosine kinase inhibitor; IL-21; pegylated IFN-α2b; axitinib (INLYTA®, Pfizer, Inc.), a tyrosine kinase inhibitor; and trametinib (MEKINIST®, GlaxoSmithKline), a MEK inhibitor (Philips and Atkins, Int Immunol., 27(1):39-46 (2015) which is incorporated herein by reference).

It is contemplated that the anti-ANGPTL4 antibody or antigen binding fragment thereof and the additional therapeutic may be given simultaneously, in the same formulation. It is further contemplated that the anti-ANGPTL4 antibody and the additional therapeutic are administered in separate formulations and administered concurrently, with concurrently referring to agents given within 30 minutes of each other.

In another aspect, the anti-ANGPTL4 antibody or antigen binding fragment thereof is administered prior to administration of the additional therapeutic. Prior administration refers to administration of the anti-ANGPTL4 antibody within the range of one week prior to treatment with the additional therapeutic up to 30 minutes before administration of the additional therapeutic. It is further contemplated that the anti-ANGPTL4 antibody is administered subsequent to administration the additional therapeutic. Subsequent administration is meant to describe administration from 30 minutes after administration of the additional therapeutic to up to one week after administration of the additional therapeutic.

It is further contemplated that other adjunct therapies may be administered, where appropriate. For example, the patient may also be administered surgical therapy, chemotherapy, a cytotoxic agent, photodynamic therapy or radiation therapy where appropriate.

Chemotherapeutic agents contemplated for use with the agents of the present disclosure include, but are not limited to, those listed in Table 1:

TABLE 1

| | |
|---|---|
| Alkylating agents | Natural products |
| Nitrogen mustards | Antimitotic drugs |
| mechlorethamine | Taxanes |
| cyclophosphamide | paclitaxel |
| ifosfamide | Vinca alkaloids |
| melphalan | vinblastine (VLB) |
| chlorambucil | vincristine |
| Nitrosoureas | vinorelbine |
| carmustine (BCNU) | Taxotere ® (docetaxel) |
| lomustine (CCNU) | estramustine |
| semustine (methyl-CCNU) | estramustine phosphate |
| Ethylenimine/Methyl-melamine | Epipodophylotoxins |
| thriethylenemelamine (TEM) | etoposide |
| triethylene thiophosphoramide | teniposide |
| (thiotepa) | Antibiotics |
| hexamethylmelamine | actimomycin D |
| (HMM, altretamine) | daunomycin (rubido-mycin) |
| Alkyl sulfonates | doxorubicin (adria-mycin) |
| busulfan | mitoxantroneidarubicin |
| Triazines | bleomycin |
| dacarbazine (DTIC) | splicamycin (mithramycin) |
| Antimetabolites | mitomycinC |
| Folic Acid analogs | dactinomycin |
| methotrexate | aphidicolin |
| Trimetrexate | Enzymes |
| Pemetrexed | L-asparaginase |
| (Multi-targeted antifolate) | L-arginase |
| Pyrimidine analogs | Radiosensitizers |
| 5-fluorouracil | metronidazole |
| fluorodeoxyuridine | misonidazole |
| gemcitabine | desmethylmisonidazole |
| cytosine arabinoside | pimonidazole |
| (AraC, cytarabine) | etanidazole |
| 5-azacytidine | nimorazole |
| 2,2′- difluorodeoxy-cytidine | RSU 1069 |
| Purine analogs | EO9 |
| 6-mercaptopurine | RB 6145 |
| 6-thioguanine | SR4233 |
| azathioprine | nicotinamide |
| 2′-deoxycoformycin | 5-bromodeozyuridine |
| (pentostatin) | 5-iododeoxyuridine |
| erythrohydroxynonyl-adenine | bromodeoxycytidine |
| (EHNA) | Miscellaneous agents |
| fludarabine phosphate | Platinium coordination complexes |
| 2-chlorodeoxyadenosine | cisplatin |
| (cladribine, 2-CdA) | Carboplatin |
| Type I Topoisomerase Inhibitors | oxaliplatin |
| camptothecin | Anthracenedione |
| topotecan | mitoxantrone |
| irinotecan | Substituted urea |
| Biological response modifiers | hydroxyurea |
| G-CSF | Methylhydrazine derivatives |
| GM-CSF | N-methylhydrazine (MIH) |
| Differentiation Agents | procarbazine |
| retinoic acid derivatives | Adrenocortical suppressant |
| Hormones and antagonists | mitotane (o, p′ - DDD) |
| Adrenocorticosteroids/antagonists | ainoglutethimide |
| prednisone and equivalents | Cytokines |
| dexamethasone | interferon (α, β, γ) |
| ainoglutethimide | interleukin-2 |
| Progestins | Photosensitizers |
| hydroxyprogesterone caproate | hematoporphyrin derivatives |
| medroxyprogesterone acetate | Photofrin ® |
| megestrol acetate | benzoporphyrin derivatives |
| Estrogens | Npe6 |
| diethylstilbestrol | tin etioporphyrin (SnET2) |
| ethynyl estradiol/equivalents | pheoboride-a |
| Antiestrogen | bacteriochlorophyll-a |
| tamoxifen | naphthalocyanines |
| Androgens | phthalocyanines |
| testosterone propionate | zinc phthalocyanines |
| fluoxymesterone/equivalents | Radiation |
| Antiandrogens | X-ray |
| flutamide | ultraviolet light |
| gonadotropin-releasing | gamma radiation |
| hormone analogs | visible light |
| leuprolide | infrared radiation |
| Nonsteroidal antiandrogens | microwave radiation |
| flutamide | |

### Pharmaceutical Compositions

For all protein-based therapeutics described herein (e.g., antibodies) administration by the delivery of gene expression constructs are contemplated as one embodiment. Any suitable vector may be used to introduce a polynucleotide that encodes a protein-based therapeutic described herein, into the host. Exemplary vectors that have been described in the literature include replication deficient retroviral vectors, including but not limited to lentivirus vectors [Kim et al., J. Virol., 72(1): 811-816 (1998); Kingsman & Johnson, Scrip Magazine, October, 1998, pp. 43 46.]; adeno-associated viral (AAV) vectors [U.S. Pat. Nos. 5,474,935; 5,139,941; 5,622,856; 5,658,776; 5,773,289; 5,789,390; 5,834,441; 5,863,541; 5,851,521; 5,252,479; Gnatenko et al., J. Invest. Med., 45: 87 98 (1997)]; adenoviral (AV) vectors [See, e.g., U.S. Pat. Nos. 5,792,453; 5,824,544; 5,707,618; 5,693,509; 5,670,488; 5,585,362; Quantin et al., Proc. Natl. Acad. Sci. USA, 89: 2581 2584 (1992); Stratford Perricadet et al., J. Clin. Invest., 90: 626 630 (1992); and Rosenfeld et al., Cell, 68: 143 155 (1992)]; an adenoviral adeno-associated viral chimeric (see for example, U.S. Pat. No. 5,856,152) or a vaccinia viral or a herpesviral (see for example, U.S. Pat. Nos. 5,879,934; 5,849,571; 5,830,727; 5,661,033; 5,328,688; Lipofectin mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in their entirety.

Pharmaceutical compositions comprising an anti-ANGPTL4 antibody or antigen binding fragment thereof described herein are also contemplated. In some embodiments, the pharmaceutical composition contains formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, methionine or lysine); antimicrobials; antioxidants (such as reducing agents, oxygen/free-radical scavengers, and chelating agents (e.g., ascorbic acid, EDTA, sodium sulfite or sodium hydrogen-sulfite)); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter-ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

Selection of the particular formulation materials described herein may be driven by, for example, the intended route of administration, delivery format and desired dosage. See, for example, REMINGTON'S PHARMACEUTICAL SCIENCES, supra. The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments, the composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in some embodiments, the antibody or (antigen binding fragment thereof) may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the composition may be provided in the form of a pyrogen-free, parenterally acceptable aqueous solution comprising the desired antibody or fragment in a pharmaceutically acceptable vehicle. A particularly suitable vehicle for parenteral injection is sterile distilled water in which the antibody or fragment is formulated as a sterile, isotonic solution, properly preserved. In certain embodiments, implantable drug delivery devices may be used to introduce the desired antibody (or antigen binding fragment thereof).

Additional pharmaceutical compositions will be evident to those skilled in the art, including formulations involving antigen binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP133988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP036676; EP088046 and EP143949, incorporated by reference.

Embodiments of the antibody formulations can further comprise one or more preservatives.

Administration of the compositions described herein will be via any common route so long as the target tissue is available via that route. The pharmaceutical compositions may be introduced into the subject by any conventional method, e.g., by intravenous, intradermal, intramuscular, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site.

### Dosage

In some embodiments, one or more doses of the antibody or antigen binding fragment are administered in an amount for a time effective to reduce or inhibit aberrant neovascularization associated with elevated levels of ANGPTL4 in a subject. In some embodiments, one or more doses of the antibody or antigen binding fragment are administered in an amount and for a time effective to treat cancer or reduce tumor burden in a subject. For example, one or more administrations of an antibody or antigen binding fragment thereof described herein are optionally carried out over a therapeutic period of, for example, about 1 week to about 24 months (e.g., about 1 month to about 12 months, about 1 month to about 18 months, about 1 month to about 9 months or about 1 month to about 6 months or about 1 month to about 3 months). In some embodiments, a subject is administered one or more doses of an antibody or fragment thereof described herein over a therapeutic period of, for example about 1 month to about 12 months (52 weeks) (e.g., about 2 months, about 3 months, about 4 months, about 5 months, about 6 months, about 7 months, about 8 months, about 9 months, about 10 months, or about 11 months).

It may be advantageous to administer multiple doses of the antibody or antigen binding fragment at a regular interval, depending on the therapeutic regimen selected for a particular subject. In some embodiments, the antibody or fragment thereof is administered periodically over a time period of one year (12 months, 52 weeks) or less (e.g., 9 months or less, 6 months or less, or 3 months or less). In this regard, the antibody or fragment thereof is administered to the human once every about 3 days, or about 7 days, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 6 months, or 12 months.

In various embodiments, one or more doses comprising from about 50 milligrams to about 1,000 milligrams of the antibody or antigen binding fragment thereof are administered to a subject (e.g., a human subject). For example, a dose can comprise at least about 5 mg, at least about 15 mg, at least about 25 mg, at least about 50 mg, at least about 60 mg, at least about 70 mg, at least about 80 mg, at least about 90 mg, at least about 100 mg, at least about 120 mg, at least about 150 mg, at least about 200 mg, at least about 210 mg, at least about 240 mg, at least about 250 mg, at least about 280 mg, at least about 300 mg, at least about 350 mg, at least about 400 mg, at least about 420 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg or up to about 1,000 mg of antibody. Ranges between any and all of these endpoints are also contemplated, e.g., about 50 mg to about 80 mg, about 70 mg to about 140 mg, about 70 mg to about 270 mg, about 75 mg to about 100 mg, about 100 mg to about 150 mg, about 140 mg to about 210 mg, or about 150 mg to about 200 mg, or about 180 mg to about 270 mg. The dose is administered at any interval, such as multiple times a week (e.g., twice or three times per week), once a week, once every two weeks, once every three weeks, or once every four weeks.

In some embodiments, the one or more doses can comprise between about 0.1 to about 50 milligrams (e.g., between about 5 and about 50 milligrams), or about 1 to about 100 milligrams, of antibody (or antigen binding fragment thereof) per kilogram of subject body weight (mg/kg). For example, the dose may comprise at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 20 mg/kg, at least about 25 mg/kg, at least about 26 mg/kg, at least about 27 mg/kg, at least about 28 mg/kg, at least about 29 mg/kg, at least about 30 mg/kg, at least about 31 mg/kg, at least about 32 mg/kg, at least about 33 mg/kg, at least about 34 mg/kg, at least about 35 mg/kg, at least about 36 mg/kg, at least about 37 mg/kg, at least about 38 mg/kg, at least about 39 mg/kg, at least about 40 mg/kg, at least about 41 mg/kg, at least about 42 mg/kg, at least about 43 mg/kg, at least about 44 mg/kg, at least about 45 mg/kg, at least about 46 mg/kg, at least about 47 mg/kg, at least about 48 mg/kg, at least about 49 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or up to about 100 mg/kg. Ranges between any and all of these endpoints are also contemplated, e.g., about 1 mg/kg to about 3 mg/kg, about 1 mg/kg to about 5 mg/kg, about 1 mg/kg to about 8 mg/kb, about 3 mg/kg to about 8 mg·kg, about 1 mg/kg to about 10 mg/kg, about 1 mg/kg to about 20 mg/kg, about 1 mg/kg to about 40 mg/kg, about 5 mg/kg to about 30 mg/kg, or about 5 mg/kg to about 20 mg/kg.

Kits

Once a pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the disclosure may each contain both a first container having a dried protein and a second container having an aqueous formulation. In certain embodiments, kits containing single and multi-chambered pre-filled syringes (e.g., liquid syringes and lyosyringes) are provided.

The following Examples are provided to further illustrate aspects of the disclosure, and are not meant to constrain the disclosure to any particular application or theory of operation.

### EXAMPLES

### Example 1—Antibody Generation and Purification

Five monoclonal rat-anti-human ANGPTL4 antibodies (Ab-A comprising CDR amino acid sequence set forth in SEQ ID NOs: 3-8, Ab-B comprising CDR amino acid sequence set forth in SEQ ID NOs: 13-18, Ab-C comprising CDR amino acid sequence set forth in SEQ ID NOs: 23-28, Ab-D comprising CDR amino acid sequence set forth in SEQ ID NOs: 33-38 and Ab-E comprising CDR amino acid sequence set forth in SEQ ID NOs: 43-48) generated against the C-Terminus fibrinogen-like signaling peptide (referred as cANGPTL4), amino acids 166-406 of SEQ ID NO: 1 (Genbank Accession No. Q9BY76).

The antibodies were generated by GenScript in Wistar rats immunized with recombinant human cANGPTL4 using service SC1717 MonoExpress™ Basic (3 rats) following standard immunization and boosting protocols. Cell fusion was performed using electrofusion of splenocytes and myeloma type SP2/0. Supernatants of hybridoma cell lines were screened for cANGPTL4 binding using direct ELISA and positive clones were sent to us for further testing on Western Blotting, Immunoprecipitation, and Blocking angiogenesis. Five hybridoma cell lines were provided to us for antibody production from hybridoma supernatants and further purified using Protein G affinity purification following standard protocols. The hybridoma cells were cultured in 2 liter rolling bottles. When 90% of the cells were dead the supernatant was collected and filtered to remove any cells and cellular debris. The filtered supernatant was passed through an affinity chromatography column packed with Protein G sepharose. The column was washed and the antibody was eluted with 0.1M glycine pH 3.0. The elution buffer was neutralized and the buffer exchanged by dialysis with PBS

### Example 2—Antibody Affinity to cANGPTL4

The kinetic binding parameters and affinity of the purified antibodies was determined by surface plasmon resonance (SPR) using Reichert 2SPR machine (Reichert Technologies) and purified recombinant human cANGPTL4 (Table 2).

TABLE 2
Summary of kinetic and affinity parameters for the
five monoclonal rat-anti-human cANGPTL4 antibodies
as determined by surface plasmon resonance.

| | Kinetic and Affinity Parameters | | |
|---|---|---|---|
| Antibody | kₐ₁ (M⁻¹s⁻¹) | k_{d1} (s⁻¹) | K_{D1}(pM) |
| Ab-B | 2.69e5 | 3.69e−5 | 132 |
| Ab-C | 6.78e4 | 1.35e−5 | 199 |
| Ab-D | 8.11e4 | 3.89e−5 | 480 |
| Ab-E | 2.15e4 | 8.07e−5 | 3740 |
| Ab-A | — | — | — |

### Example 3—Inhibition of cANGPTL4-Induced Angiogenesis In Vitro and In Vivo

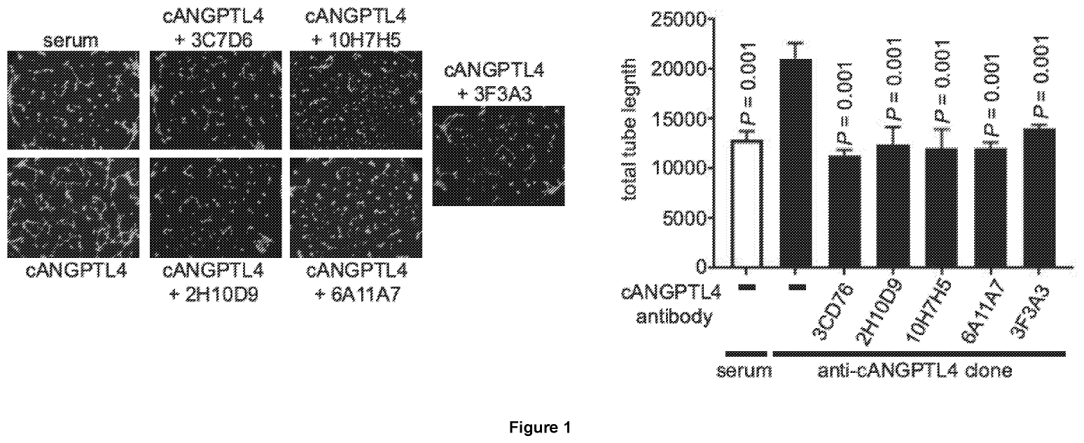

To assess the ability of the purified antibodies to inhibit cANGPTL4-induced angiogenesis, an in vitro endothelial cell tube formation assay was conducted. Endothelial cells were plated in 3D culture on Matrigel and treated with recombinant 20 μg/ml human cANGPTL4 in the presence of the antibodies Ab-A, Ab-B, Ab-C, Ab-D and Ab-E or control IgG. The cells were incubated for six hours and stained with Calcein AM. The cells were visualized with a fluorescent microscope and the formation of tube-like structures were measured using AngioTool software (FIG. 1)¹.

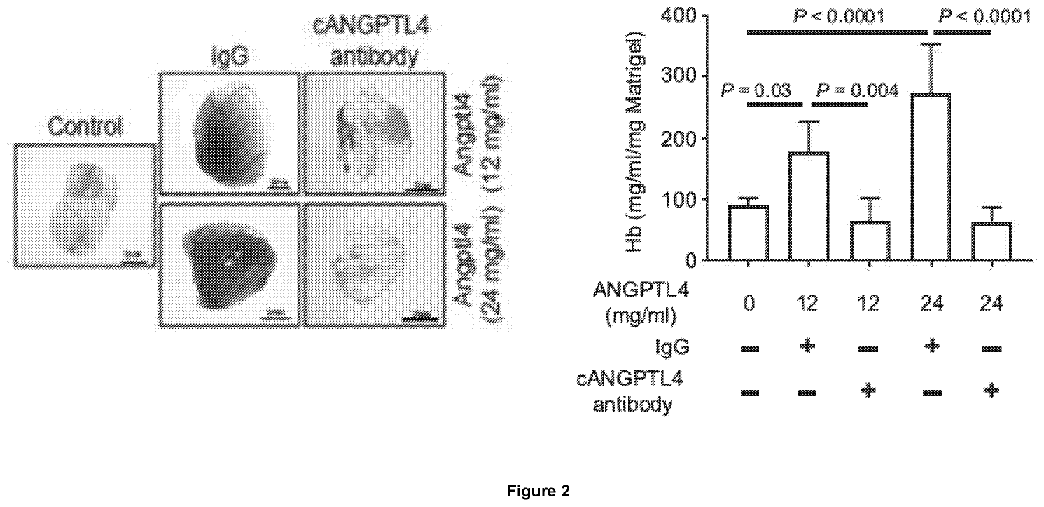

To test the ability of the antibodies to inhibit cANGPTL4 induced angiogenesis in vivo a matrigel plug assay was conducted. Growth factor reduced Matrigel (Corning) with 12 or 24 μg/ml recombinant human cANGPTL4, or without as a control, with 48 μg/ml rat IgG or our monoclonal cANGPTL4 antibody clone 6AIIA7, were implanted subcutaneously into the flank athymic nude mice. After six days, the matrigel plugs were removed, imaged and the amount of angiogenesis was assayed by measuring hemoglobin levels using a Drabkin's reagent kit (Sigma-Aldrich) (FIG. 2)¹.

### Example 4—Anti-ANGPTL4 Antibody to Treat Obesity-Driven Breast Cancer Progression in Combination with Standard-of-Care Chemotherapy

C57Bl/6 mice will be given a normal diet provided by the vivarium or a high-fat diet to induce obesity then Py8119 breast cancer cells will be implanted into the mammary gland. Mice will be randomly divided into groups and be treated with an anti-ANGPTL4 antibody described herein alone or in combination with a modified dose-dense chemotherapy regiment consisting of doxorubicin, paclitaxel and cyclophosphamide. The following groups will be used in these studies: 1) ND mock; 2) ND standard therapy (dose-dense for Py8119) 3) HFD mock; 4) HFD standard therapy; 5) HFD anti-cANGPTL4; 6) HFD standard therapy +anti-cANGPTL4. Tumor growth will be monitored and a survival curve will be generated. Tumor angiogenesis will be assessed by immunohistochemistry. These studies will provide pre-clinical data for the efficacy of targeting cANGPTL4 in obese breast cancer patients.

### Example 5—Anti-cANGPTL4 Antibody in Renal Cell Carcinoma Alone or in Combination with Anti-VEGF Therapy

RENCA renal cell carcinoma cells will be implanted into the flank of Balb/c mice. Mice will then be randomly divided into four groups and treated as follows: 1) control IgG; 2) anti-ANGPTL4 antibody described herein; 3) anti-VEGFR2 antibody (purified from DC101 hybridoma cells); 4) anti-ANGPTL4 antibody described herein+anti-VEGFR2 antibody. Tumor growth will be monitored and survival curve will be generated. Tumor angiogenesis will be assessed by immunohistochemistry. These studies will provide data for the efficacy of targeting cANGPTL4 alone or in combination with anti-VEGF therapies in RCC.

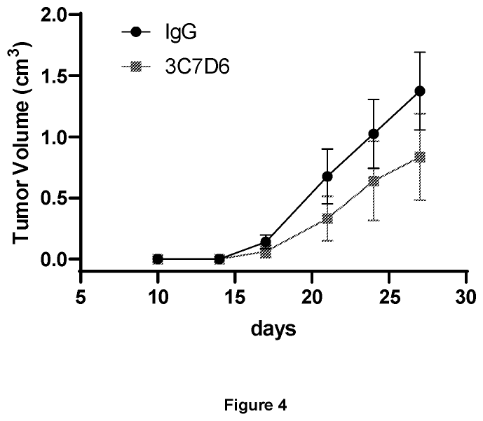

In another experiment, 10⁴ RENCA renal cell carcinoma cells were implanted into the flank (50/50 matrigel/PBS) of Balb/c mice and treated with either 100m anti-ANGPTL4 antibody or control IgG every four days starting at day 12. Tumor growth was monitored and survival curve was generated (FIG. 4). As shown in FIG. 4, tumor volume was decreased mice treated with the anti-cANGPTL4 antibody.

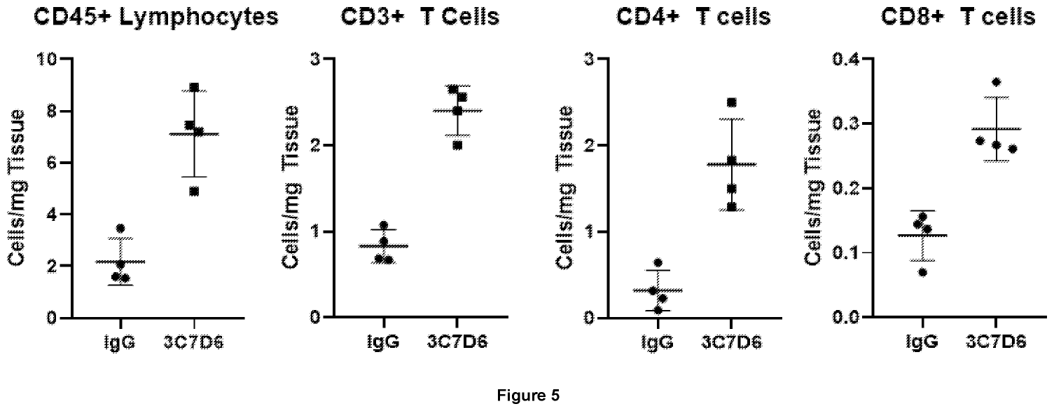

Immune cell infiltration in the tumors was assessed by flow cytometry. As shown in FIG. 5, tumors showed an increase in immune cell infiltration in mice treated with the anti-ANGPTL4 antibody. Normalization of blood vessels with anti-angiogenic therapies increased immune cell infiltration, which is an indication of potential combination with immunotherapy, which is a current strategy to treat RCC—a combination of anti-VEGFR2 small molecule inhibitors and anti-PD-1 antibody.

### Example 6—Ability of the cANGPTL4 Antibodies to Block the Inhibition of Lipoprotein Lipase Activity by Full Length ANGPTL4

ANGPTL4 is a protein consisting of an n-terminus coiled-coiled domain and a c-terminus fibrinogen domain, the amino acid sequence of which is set forth in SEQ ID NO: 1. When secreted the protein can be cleaved into two signaling peptides, the N-terminus (nANGPTL4, amino acids 1-165 of SEQ ID NO: 1 (Genbank Accession No. Q9BY76)), and the C-terminus (cANGPTL4), amino acids 166-406 of SEQ ID NO: 1 (Genbank Accession No. Q9BY76) by protein convertases. nANGPTL4 signaling peptide inhibits lipoprotein lipase (LPL) and is important in regulating lipid homeostasis. Antibodies described herein were generated against cANGPTL4 and inhibit the induction of angiogenesis by cANGPTL4. While the ANGPTL4 is often cleaved and functions as two separate signaling peptides, it can be secreted as a full length protein which can still inhibit lipoprotein lipase. Antibodies which can bind to the full length protein and block the inhibition of LPL may have unwanted side effects. Experiments will be performed to determine if anti-cANGPTL4 antibodies described herein can block the inhibition of LPL by full length ANGPTL4. Recombinant human LPL will be incubated with recombinant human full length ANGPTL4 plus anti-cANGPTL4 antibodies or rat IgG for 30 minutes. LPL activity will be assayed using EnzChek lipase fluorescent substrate and measuring fluorescence using a plate reader. These studies will elucidate whether the anti-cANGPTL4 antibodies described herein specifically inhibit the function of cleaved cANGPTL4, or if they also block the ability of full length ANGPTL4 to inhibit LPL activity. If the antibodies specifically inhibit the function of cleaved cANGPTL4 but bot full length ANGPTL4 they may have fewer unwanted side effects than antibodies which may inhibit the function of the full length protein.

### Example 7—ANGPTL4 is Upregulated in Human Renal Cell Carcinoma (RCC) Xenografts Treated with Other RCC Therapies

The first line of therapy for metastatic RCC is to use small molecule inhibitors which target VEGFR2—the receptor for VEGFA, which is an oncogenic driver for RCC. While these therapies show some efficacy, this is limited by the development of resistance in most cases¹⁴. Resistance to these therapies can be caused by upregulation of alternative angiogenic pathways, and high expression of ANGPTL4 has been correlated with poor response to VEGF targeted therapies in other systems¹⁵. A publicly available dataset, GSE64052¹⁶, was obtained with gene expression of human RCC xenografts in mice before and after development of resistance to sunitinib and sorafenib treatment and the expression of ANGPTL4 was examined.

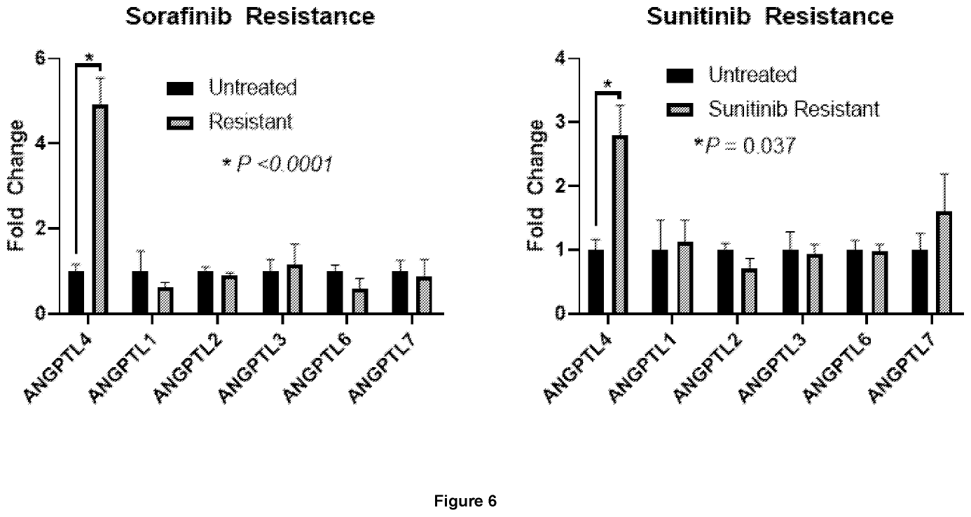

As shown in FIG. 6, ANGPTL4 is upregulated in tumors resistant to treatment with sunitinib and sorafenib, both of which are VEGFR-2 inhibitors and current therapies for RCC. The data provided in this Example demonstrates that anti-ANGPTL4 inhibitor (such as an anti-ANGPTL4 antibody described herein) would be useful in treating RCC tumors resistant to anti-VEGF therapies.

### REFERENCES CITED

1. Kolb, et al., Oncogene 2019, 38 (13), 2351-2363.
2. Tolaney, et al., Proc Natl Acad Sci USA 2015, 112 (46), 14325-30.
3. Kolb, et al., Nat Commun 2016, 7, 13007.
4. Arendt, et al., Cancer Res 2013.
5. Incio, et al., Sci Transl Med 2018, 10 (432).
6. Bal, et al., Sci Rep 2015, 5, 17717.
7. Vachhani, P.; George, S., VEGF inhibitors in renal cell carcinoma. Clin Adv Hematol Oncol 2016, 14 (12), 1016-1028.
8. Uhlen, et al., Science 2017, 357 (6352).
9. Yang, et al., Biosci Rep 2018, 38 (5).
10. Yau, et al., Diabetes Care 2012, 35 (3), 556-64.
11. Bahrami, et al., Asia Pac J Ophthalmol (Phila) 2017, 6 (6), 535-545.
12. Babapoor-Farrokhran, et al., Proc Natl Acad Sci USA 2015, 112 (23), E3030-9.
13. Kim, et al., Retina 2018, 38 (3), 523-530.
14. Mollica, et al. Cancers 2019, 11 (6), 830.
15. Bal, et al. Sci Rep 2015, 5:171717.
16. Zhang, et al. Clin Cancer Res 2015, 21 (8), 1925-1934.

What is claimed is:

1. An antibody or antigen binding fragment thereof that binds angiopoietin-like 4 (ANGPTL4) comprising amino acid sequences set forth in (a) SEQ ID NOs: 3-8, (b) SEQ ID NOs: 13-18, (c) SEQ ID NOs: 23-28, (d) SEQ ID NOs: 33-38, or (e) SEQ ID NOs: 43-48.

2. The antibody or antigen binding fragment of claim 1 that comprises a light chain variable region at least 90% identical to an amino acid sequence set forth in (a) SEQ ID NO: 10, (b) SEQ ID NO: 20, (c) SEQ ID NO: 30, (d) SEQ ID NO: 40, or (e) SEQ ID NO: 50.

3. The antibody or antigen binding fragment of claim 1 or claim 2 that comprises a light chain variable region comprising an amino acid sequence set forth in (a) SEQ ID NO: 10, (b) SEQ ID NO: 20, (c) SEQ ID NO: 30, (d) SEQ ID NO: 40, or (e) SEQ ID NO: 50.

4. The antibody or antigen binding fragment of any one of claims 1-3, that comprises a heavy chain variable region at least 90% identical to an amino acid sequence set forth in (a) SEQ ID NO: 9, (b) SEQ ID NO: 19, (c) SEQ ID NO: 29, (d) SEQ ID NO: 39, or (e) SEQ ID NO: 49.

5. The antibody or antigen binding fragment of any one of claims 1-4 that comprises a heavy chain variable region comprising an amino acid sequence set forth in (a) SEQ ID NO: 9, (b) SEQ ID NO: 19, (c) SEQ ID NO: 29, (d) SEQ ID NO: 39, or (e) SEQ ID NO: 49.

6. The antibody or antigen binding fragment thereof of any one of claims 1-5, that is a monoclonal antibody.

7. The antibody or antigen binding fragment thereof of any one of claims 1-6 that is a human or humanized antibody.

8. The antibody or antigen binding fragment thereof of any one of claims 1-7, that is an IgG.

9. The antibody or antigen binding fragment thereof of any one of claims 1-8, wherein the antigen binding fragment is a Fab fragment or an scFv.

10. A method of inhibiting ANGPLT4-induced angiogenesis in subject in need thereof, comprising administering to the subject the antibody or antigen binding fragment thereof of any one of claims 1-9.

11. The method of claim 10, wherein the subject is suffering from cancer.

12. The method of claim 10 or claim 11, wherein the subject is suffering from breast cancer or renal cell carcinoma.

13. The method of claim 12, wherein the breast cancer is basal-like breast cancer.

14. The method of any one of claims 10-13 wherein the subject is obese.

15. The method of claim 10, wherein the subject is suffering from diabetic retinopathy.

16. The method of claim 10, wherein the subject is suffering from macular age-related degeneration (AMD).

17. The method of claim 16, wherein the AMD is wet AMD.

18. The method of any one of claims 10-17, wherein the subject was hyporesponsive to prior anti-VEGF-A therapy.

19. The method of claim 18, wherein the anti-VEGF-A therapy is sorafenib, sunitinib, bevacizumab or ranibizumab.

20. A method of treating cancer in a subject in need thereof comprising administering to the subject the antibody or antigen binding fragment thereof of any one of claims 1-9.

21. The method of claim 20, wherein the subject is suffering from breast cancer or renal cell carcinoma.

22. The method of claim 21, wherein the breast cancer is basal-like breast cancer.

23. The method of any one of claims 20-22, wherein the subject is obese.

24. The method of any one of claims 20-23, further comprising administering chemotherapy to the subject.

25. A method of treating a disorder associated with elevated angiopoietin-like 4 (ANGPTL4) in a subject in need thereof comprising administering to the subject an antibody or antigen binding fragment of any one of claims 1-9 and an anti-VEGF-A therapy.

26. The method of claim 25, wherein the disorder associated with elevated ANGPTL4 is cancer, diabetic retinopathy or age-related macular degeneration (AMD).

27. The method of 26, wherein the cancer is breast cancer or renal cell carcinoma.

28. The method of claim 27, wherein the breast cancer is basal-like breast cancer.

29. The method of claim 26, wherein the age-related macular degeneration is wet AMD.

30. The method of any one of claims 25-29, wherein the subject is obese.

31. The method of any one of claims 26-30, wherein the VEGF-A therapy is sorafenib, sunitinib, bevacizumab or ranibizumab.


SEQUENCE LISTING (52 sequences)

SEQ ID NO: 1 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - protein, 241 aa, Homo sapiens
PEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 3 - protein, 5 aa, Homo sapiens
TNGVG

SEQ ID NO: 4 - protein, 16 aa, Homo sapiens
TIWAGGSTNYNSAVQS

SEQ ID NO: 5 - protein, 18 aa, Homo sapiens
QPFYYYDGIHYTRGYFDY

SEQ ID NO: 6 - protein, 11 aa, Homo sapiens
KASKSISKYLA

SEQ ID NO: 7 - protein, 7 aa, Homo sapiens
DGSTLQS

SEQ ID NO: 8 - protein, 9 aa, Homo sapiens
QQHNEYPAT

SEQ ID NO: 9 - protein, 145 aa, Homo sapiens
MAVLVLLLCLVTFPSCVLSQVQLKESGPGLMQPSETLSLTCTVSGFSLTTNGVGWVRQPLGKGLVWMGTIWAGGSTNYNSAVQSRLSISRDTSKSQVFLKMNSLQPEDTGTYYCARQPFYYYDGIHYTRGYFDYWGQGVMVTVSS

SEQ ID NO: 10 - protein, 127 aa, Homo sapiens
MRVQIQFWGLLLLWTSGIQCDVQMTQSPYNLAASPGESVSINCKASKSISKYLAWYQQKLGKANKLLIYDGSTLQSGIPSRFSGSGSGTDFTLTIRSLEPEDFGLYYCQQHNEYPATFGGGTKLELK

SEQ ID NO: 11 - DNA, 435 bp, Homo sapiens
atggctgtcctggtgctattgctctgcctggtgacatttccaagctgtgtcctgtcccaggtgcagctgaaggagtcaggacctggcttgatgcagccctcagagaccctgtccctcacctgcactgtctctggcttctcactaaccaccaatggtgtaggctgggttcgacaacctctaggaaagggtttggtgtggatgggaacaatatgggctggtggaagtacaaattataattcagctgtccaatcccgactgagcatcagcagggacacctccaagagccaagttttcttaaaaatgaacagtctgcaacctgaagacacaggcacttactactgtgccagacaacctttttattactatgatggtattcattacacgagggggtactttgattactggggccaaggagtcatggtcacagtctcctca

SEQ ID NO: 12 - DNA, 381 bp, Homo sapiens
atgagggtccagattcagttttgggggcttcttctgctctggacatcaggtatacagtgtgatgtccagatgacccagtctccatataatcttgctgcctctcctggagaaagtgtctccatcaattgcaaggcaagtaagagcattagtaagtatttagcctggtatcaacagaaacttgggaaagcaaataagcttcttatctacgatgggtcaactttgcaatctggaattccatcgaggttcagtggcagtggatctggtacagatttcactctcaccatcagaagcctggagcctgaagattttggactctattactgtcaacagcataatgaatacccagcgacgttcggtggaggcaccaagctggaattgaaa

SEQ ID NO: 13 - protein, 5 aa, Homo sapiens
NYGMA

SEQ ID NO: 14 - protein, 17 aa, Homo sapiens
SISTGGSDTYYRDSVKG

SEQ ID NO: 15 - protein, 9 aa, Homo sapiens
QGFTEDTDY

SEQ ID NO: 16 - protein, 11 aa, Homo sapiens
KASQNINKYLN

SEQ ID NO: 17 - protein, 7 aa, Homo sapiens
NTNNLQT

SEQ ID NO: 18 - protein, 9 aa, Homo sapiens
LQHNSFPGT

SEQ ID NO: 19 - protein, 137 aa, Homo sapiens
MDFRLSLAFLVLLIKGVQCEVQLVESGGGLVQPGRSLKLSCAASGFTFSNYGMAWVRQTPTKGLEWVASISTGGSDTYYRDSVKGRFTISRDNAKNTQYLQMDSLRSEDTATYYCARQGFTEDTDYWGQGVMVTVSS

SEQ ID NO: 20 - protein, 126 aa, Homo sapiens
MAPVQLLGLLLIWLPAMRCDIQMTQSPSFLSASVGDRVTINCKASQNINKYLNWYQQKLGEAPKRLIYNTNNLQTGIPSRFSGSGSGTDYTLTISSLQPEDFATYFCLQHNSFPGTFGGGTKLELK

SEQ ID NO: 21 - DNA, 411 bp, Homo sapiens
atggacttcaggctcagcttggctttccttgtccttttaataaaaggtgtccagtgtgaggtgcagctggtggagtctgggggaggcttagtgcagcctggaaggtccctgaaactctcctgtgcagcctcaggattcactttcagtaactatggcatggcctgggtccgccagactccaacgaagggtctggagtgggtcgcatccattagtactggtggtagtgacacttactatcgagactccgtgaagggccgattcactatctccagagataatgcaaaaaacacccaatacttgcaaatggacagtctgaggtctgaggacacggccacttattactgtgcaagacagggctttacggaggataccgattactggggccaaggagtcatggtcacagtctcctca

SEQ ID NO: 22 - DNA, 378 bp, Homo sapiens
atggctccagtccagctcttagggctgctgctgatttggctcccagccatgagatgtgacatccagatgacccagtctccttcattcctgtctgcatctgtgggagacagagtcactatcaactgcaaagcaagtcagaatattaacaagtacttaaactggtatcagcaaaagcttggagaagctcccaaacgcctgatatataatacaaacaatttgcaaacaggcatcccatcaaggttcagtggcagtggatctggtacagattacacactcaccatcagcagcctgcagcctgaagattttgccacatatttctgcttgcagcataatagttttcccgggacgttcggtggaggcaccaagctggaattgaaa

SEQ ID NO: 23 - protein, 5 aa, Homo sapiens
NYGMA

SEQ ID NO: 24 - protein, 17 aa, Homo sapiens
SISSGGGNTYYRDSVKG

SEQ ID NO: 25 - protein, 9 aa, Homo sapiens
DPGITGFDY

SEQ ID NO: 26 - protein, 11 aa, Homo sapiens
KASQNINKYLN

SEQ ID NO: 27 - protein, 7 aa, Homo sapiens
NTNTLQT

SEQ ID NO: 28 - protein, 9 aa, Homo sapiens
LQHNSFPGT

SEQ ID NO: 29 - protein, 137 aa, Homo sapiens
MDFRLSLAFLVLLIKGVQCEVQLVDSGGGLVQPGRSLKLSCAASGFTFSNYGMAWVRQIPTKGLEWVASISSGGGNTYYRDSVKGRFTISRDNAKNTQYLQMDSLRSEDTATYYCARDPGITGFDYWGQGVMVTVSS

SEQ ID NO: 30 - protein, 126 aa, Homo sapiens
MAPVQLLGLLLIWLPAMRCDIQMTQSPSFLSASVGDRVTINCKASQNINKYLNWYQQKLGEAPKRLIYNTNTLQTGIPSRFSGSGSGTDYTLTISSLQPEDFATYFCLQHNSFPGTFGPGTTLELK

SEQ ID NO: 31 - DNA, 411 bp, Homo sapiens
atggacttcaggctcagcttggctttccttgtccttttaataaaaggtgtccagtgtgaggtgcagctggtggactctgggggaggcttagtgcagcctggaaggtccctgaaactctcctgtgcagcctcaggattcactttcagtaactatggcatggcctgggtccgccagattccaacgaagggtctggagtgggtcgcatccattagtagtggtggtggtaacacttactatcgagactccgtgaagggccgattcactatctccagagataatgcaaaaaacacccaatacttgcaaatggacagtctgaggtctgaggacacggccacttattactgtgcaagagacccgggtataaccggctttgattactggggccaaggagtcatggtcacagtctcctca

SEQ ID NO: 32 - DNA, 378 bp, Homo sapiens
atggctccagtccagctcttagggctgctgctgatttggctcccagccatgagatgtgacatccagatgacccagtctccttcattcctgtctgcatctgtgggagacagagtcactatcaactgcaaagcaagtcagaatattaacaagtacttaaactggtatcagcaaaagcttggagaagctcccaaacgcctgatatataatacaaacactttgcaaacaggcatcccatcaaggttcagtggcagtggatctggtacagattacacactcaccatcagcagcctgcagcctgaagattttgccacatatttctgcttgcagcataatagttttcccggcacgtttggacctgggaccacgctggaactgaaa

SEQ ID NO: 33 - protein, 5 aa, Homo sapiens
TNGVG

SEQ ID NO: 34 - protein, 17 aa, Homo sapiens
SISTGGGNTYYRDSVKG

SEQ ID NO: 35 - protein, 9 aa, Homo sapiens
DSTVDYFDY

SEQ ID NO: 36 - protein, 11 aa, Homo sapiens
KASQKINKYLN

SEQ ID NO: 37 - protein, 7 aa, Homo sapiens
NTNNLQT

SEQ ID NO: 38 - protein, 9 aa, Homo sapiens
LQHNSFPGT

SEQ ID NO: 39 - protein, 137 aa, Homo sapiens
MDFRLSLAFLVLLIKGVQCEVQLVESGGGLVQPGRSLKLSCAASGFTFSNYGMAWVRQTPTKGLEWVASISTGGGNTYYRDSVKGRFTISRDNAKNTQYLQMDSLRSEDTATYYCARDSTVDYFDYWGQGVMVTVSS

SEQ ID NO: 40 - protein, 126 aa, Homo sapiens
MAPVQLLGLLLIWLPAMRCDIQMTQSPSFLSASVGDRVTINCKASQKINKYLNWYQQKLGEAPKRLIYNTNNLQTGIPSRFSGSGSGTDFTLTISSLQPEDFATYCCLQHNSFPGTFGGGTKLELK

SEQ ID NO: 41 - DNA, 411 bp, Homo sapiens
atggacttcaggctcagcttggctttccttgtccttttaataaaaggtgtccagtgtgaggtgcagctggtggagtctgggggaggcttagtgcagcctggaaggtccctgaaactctcctgtgcagcctcaggattcactttcagtaactatggcatggcctgggtccgccagactccaacgaagggtctggagtgggtcgcatccattagtactggtggtggtaacacttactatcgagactccgtgaagggccgattcactatctccagagataatgcaaaaaacacccaatacttgcaaatggacagtctgaggtctgaggacacggccacttattactgtgcaagagatagtacagtggactactttgattactggggccaaggagtcatggtcacagtctcctca

SEQ ID NO: 42 - DNA, 378 bp, Homo sapiens
atggctccagtccagctcttagggctgctgctgatttggctcccagccatgagatgtgacatccagatgacccagtctccttcattcctgtctgcatctgtgggagacagagtcactatcaactgcaaagcaagtcagaagattaacaagtacttaaactggtatcagcaaaagcttggagaagctcccaaacgcctgatatataatacaaacaatttgcaaacaggcatcccatcaaggttcagtggcagtggatctggtacagatttcacactcaccatcagcagcctgcagcctgaagattttgccacatattgttgcttgcagcataatagttttcccgggacgttcggtggaggcaccaagctggaattgaaa

SEQ ID NO: 43 - protein, 5 aa, Homo sapiens
SFAMA

SEQ ID NO: 44 - protein, 17 aa, Homo sapiens
SISYDGGNTYYRDSVKG

SEQ ID NO: 45 - protein, 10 aa, Homo sapiens
ADLIWWYFDF

SEQ ID NO: 46 - protein, 11 aa, Homo sapiens
QASQDIGNWLA

SEQ ID NO: 47 - protein, 7 aa, Homo sapiens
GSTSLAD

SEQ ID NO: 48 - protein, 9 aa, Homo sapiens
QQASSAPPT

SEQ ID NO: 49 - protein, 138 aa, Homo sapiens
MDIRLSLAFLVLFIKGVQCEVQLVESGGGLVQPGRSMKLSCAASGFTFSSFAMAWVRQAPTKGLEWVASISYDGGNTYYRDSVKGRFTISRDNAKSSLYLQMDSLRSEDTSTYYCASADLIWWYFDFWGPGTMVTVSS

SEQ ID NO: 50 - protein, 127 aa, Homo sapiens
MNVPTQLLGLLILWLTGGKCDIQMTQSPASLSASLEEIVTITCQASQDIGNWLAWYQQKPGKSPQLLIYGSTSLADGVPSRFSGSRSGTQYSLKISRLQVEDIGIYYCQQASSAPPTFGAGTKLELK

SEQ ID NO: 51 - DNA, 414 bp, Homo sapiens
atggacatcaggctcagcttggctttccttgtccttttcataaaaggtgtccagtgtgaggtgcagctggtggagtctgggggaggcttagtgcagcctggaaggtccatgaaactctcctgtgcagcctctggattcactttcagtagctttgcaatggcctgggtccgccaggctccaacgaaggggctggagtgggtcgcatccattagttatgatggtggtaacacttactatcgagactccgtgaagggccgattcactatctccagagataatgcaaaaagcagcctatacctgcaaatggacagtctgaggtctgaggacacgtccacttattactgtgcaagcgccgatcttatctggtggtactttgacttctggggcccaggaaccatggtcaccgtgtcctca

SEQ ID NO: 52 - DNA, 381 bp, Homo sapiens
atgaatgtgcccactcaactccttgggttgctgatactctggcttacaggtggtaaatgtgacatccagatgacacagtctcctgcctccctgtctgcatctctggaagaaattgtcaccatcacatgccaggcaagccaggacattggtaattggttagcatggtatcagcagaaaccggggaaatctcctcagctcctgatctatggttcaacaagcttggcagatggagtcccatcaaggttcagcggcagtagatctggcacacagtattcccttaagatcagcagactacaggttgaagatattggaatctactactgtcaacaggcttctagtgctcctccaacgtttggagctgggaccaagctggaactgaaa